(19) United States
(12) Patent Application Publication
(10) Pub. No.: US 2017/0215932 A1
(43) Pub. Date: Aug. 3, 2017

(54) ADJUSTABLE IMPLANT FOR FIXING FEMUR

(71) Applicant: GSM KOREA CO., LTD., Gwangju (KR)

(72) Inventors: Jung-Won SHIN, Gwangju (KR); Gyeong-Su KIM, Seoul (KR); Soo-Cheol LEE, Seoul (KR)

(21) Appl. No.: 15/341,882

(22) Filed: Nov. 2, 2016

(30) Foreign Application Priority Data
Feb. 1, 2016 (KR) 10-2016-0012145

(51) Int. Cl. A61B 17/80; A61B 17/84; A61B 17/82

(52) U.S. Cl. CPC A61B 17/8085; A61B 17/82; A61B 17/8061; A61B 17/842

(57) ABSTRACT

Provided is an adjustable implant for fixing a femur, the adjustable implant allowing a wire wound around a fractured femur to be accurately placed at a desired location even when the desired location is changed depending on a patient. The adjustable implant includes: a body (100) provided with first locking holes (110) vertically penetrating therethrough at predetermined intervals; a head (200) extending from an upper end of the body (100), and being bent to be in a hook shape for being hooked over a greater trochanter of a femur; and a detachable wire locking part (300) detachably mounted to the body (100) or to the head (200), and supporting a wire (400), wherein the detachable wire locking part (300) includes: base parts (310); and a wire locking member (320) having a coupling member (321) so as to be detachably coupled to each of the base parts (310).

### CROSS REFERENCE

This application claims foreign priority under Paris Convention to Korean Patent Application No. 10-2016-0012145, filed 2 Feb. 2016, with the Korean Intellectual Property Office.

### BACKGROUND

The present invention relates to an adjustable implant for fixing a femur, the adjustable implant allowing a wire wound around a fractured femur to be accurately placed at a desired location even when the desired location is changed depending on a patient.

Generally, when the greater trochanter or the lesser trochanter of the femur is broken, in order to treat the fractured segment, a wire, a bone plate using screws, or both are used to fix the fractured bone.

However, in the case where the wire is used to fix the fractured bone, it is problematic in that the wire may deviate from a target area in the process of winding the wire around the bone. Further, as time passes, the wire may become unwound or may move.

In the case where the bone plate is used, the screws should be driven into the bone to fix the bone plate to the bone, but in the case where the fractured bone is weak, the bone plate may not be used.

Further, in the case where both the wire and the bone plate are used simultaneously, the wire may not be provided in a desirable area or the wire may become unwound after a procedure.

The foregoing is intended merely to aid in the understanding of the background of the present invention, and is not intended to mean that the present invention falls within the purview of the related art that is already known to those skilled in the art.

### SUMMARY OF THE INVENTION

Accordingly, the present invention has been made keeping in mind the above problems occurring in the related art, and the present invention is intended to propose an adjustable implant for fixing a femur, the adjustable implant allowing a wire wound around a fractured femur to be accurately placed at a desired location even when the desired location is changed depending on a patient.

The present invention is further intended to propose an adjustable implant for fixing a femur, the adjustable implant capable of preventing a wire from being separated therefrom by having contact protrusions provided on each of inner surfaces of through-holes of a wire locking member, by which the wire is locked when the wire is placed therethrough.

The present invention is further intended to propose an adjustable implant for fixing a femur, the adjustable implant allowing the through-holes of the wire locking member to come into close contact with the wire by applying small pressure to the through-holes. This is realized by providing slits extending from an upper surface of the wire locking member locking the wire when the wire is placed.

The present invention is furthermore intended to propose an adjustable implant for fixing a femur, the adjustable implant capable of adjusting a gap between a head disposed over a greater trochanter of a femur, and a body longitudinally disposed over the femur, and thereby the adjustable implant can be applied to various femur sizes.

In order to achieve the above object, according to one aspect of the present invention, there is provided an adjustable implant for fixing a femur, the adjustable implant including: a body 100 provided with first locking holes 110 vertically penetrating therethrough at predetermined intervals; a head 200 extending from an upper end of the body 100, and being bent to be in a hook shape for being hooked over a greater trochanter of a femur; and a detachable wire locking part 300 detachably mounted to the body 100 or to the head 200, and supporting a wire 400, wherein the detachable wire locking part 300 includes: base parts 310 provided on each of opposite sides of an upper surface of the body 100 or of a bent part 210 at predetermined intervals; and a wire locking member 320 being in a hexahedral shape, and having a coupling member 321 on a lower surface thereof so as to be detachably coupled to each of the base parts 310, with a pair of through-holes 322 penetrating through opposite sides of the wire locking member.

In order to achieve the above object, according to another aspect of the present invention, there is provided an adjustable implant for fixing a femur, the adjustable implant including: a body 100 including: first locking holes 110 provided by vertically penetrating therethrough at predetermined intervals; insertion grooves 150 provided at an upper end of the body; and locking grooves 160 provided on side surfaces of the body 100 and extending toward the insertion grooves 150; a head 200 being bent to be in a hook shape for being hooked over a greater trochanter of a femur, and including connection protrusions 250 protrudingly provided at a lower end thereof so as to be engaged with the insertion grooves 150 of the body, with space adjusting grooves 260 being provided in the connection protrusions 250 at predetermined intervals; and a detachable wire locking part 300 detachably mounted to the body 100 or to the head 200, and supporting a wire 400, wherein the detachable wire locking part 300 includes: base parts 310 provided on each of opposite sides of an upper surface of the body 100 or of a bent part 210 at predetermined intervals; and a wire locking member 320 being in a hexahedral shape, and having a coupling member 321 on a lower surface thereof so as to be detachably coupled to each of the base parts 310, with a pair of through-holes 322 penetrating through opposite sides of the wire locking member.

Preferably, the body 100 is configured such that a lower surface thereof is in a curved shape so as to allow opposite edges of the lower surface come into close contact with a surface of a femur.

Preferably, the head 200 includes: a bent part 210 extending from the upper end of the body 100 and being gently bent so as to come into contact with the greater trochanter of the femur; a hook 220 provided at an end of the bent part 210 to lock the implant by being hooked over the greater trochanter of the femur; and a fixing protrusion 230 provided on a rear surface of the bent part 210, and coming into close contact with a femur, thereby preventing the implant from moving on the femur.

Preferably, the bent part 210 is provided with second locking holes 211 vertically penetrating therethrough at predetermined intervals.

Preferably, the base part 310 of the detachable wire locking part 300 includes a plurality of seat grooves being in a cylindrical shape and provided at predetermined intervals; and the coupling member 321 of the wire locking member 320 includes protrusions being in a cylindrical shape to be engaged with the seat grooves.

Preferably, each of the body 100 and the bent part 210 is provided with a support part 330 on the upper surface thereof by protruding upward along a periphery of the base part 310 so as to support the opposite sides of the wire locking member 320.

Preferably, contact protrusions 500 protrude on each of inner surfaces of the through-holes 322 of the wire locking member 320.

Preferably, the wire locking member 320 is provided with slits 600 extending from an upper surface of the wire locking member to the through-holes 322.

The adjustable implant for fixing a femur is capable of adjusting a location of a wire wound around a fractured femur, thereby allowing the wire to be accurately placed at a desired location.

Further, the adjustable implant for fixing a femur is capable of preventing the wire from being separated therefrom since the contact protrusions are provided on each of inner surfaces of through-holes of the wire locking member.

Further, the adjustable implant for fixing a femur allows the through-holes of the wire locking member to come into close contact with the wire by applying small pressure, by providing slits extending from an upper surface of the wire locking member to the through-holes.

Further, the adjustable implant for fixing a femur is capable of being applied to various femur sizes since a gap between the head and the body is adjustable.

### BRIEF DESCRIPTION OF THE DRAWINGS

The above and other objects, features and other advantages of the present invention will be more clearly understood from the following detailed description when taken in conjunction with the accompanying drawings, in which:

FIG. 1A is a perspective front view showing an adjustable implant for fixing a femur according to a first embodiment of the present invention;

FIG. 1B is a perspective rear view showing the adjustable implant for fixing a femur according to the first embodiment of the present invention;

FIG. 2 is an exemplary view showing a mounting state of a detachable wire locking part according to the present invention;

FIG. 3 is an exemplary view showing a wire locking member formed with contact protrusions according to an embodiment of the present invention;

FIG. 4 is an exemplary view showing the wire locking member formed with slits according to an embodiment of the present invention;

FIG. 5 is an exemplary view showing a state where the adjustable implant for fixing a femur according to the present invention is used; and

FIG. 6 is a perspective front view showing an adjustable implant for fixing a femur according to a second embodiment of the present invention.

### DETAILED DESCRIPTION OF THE INVENTION

Hereinbelow, exemplary embodiments of the present invention will be described in detail with reference to the accompanying drawings. Throughout the drawings, the same reference numerals will refer to the same or like parts.

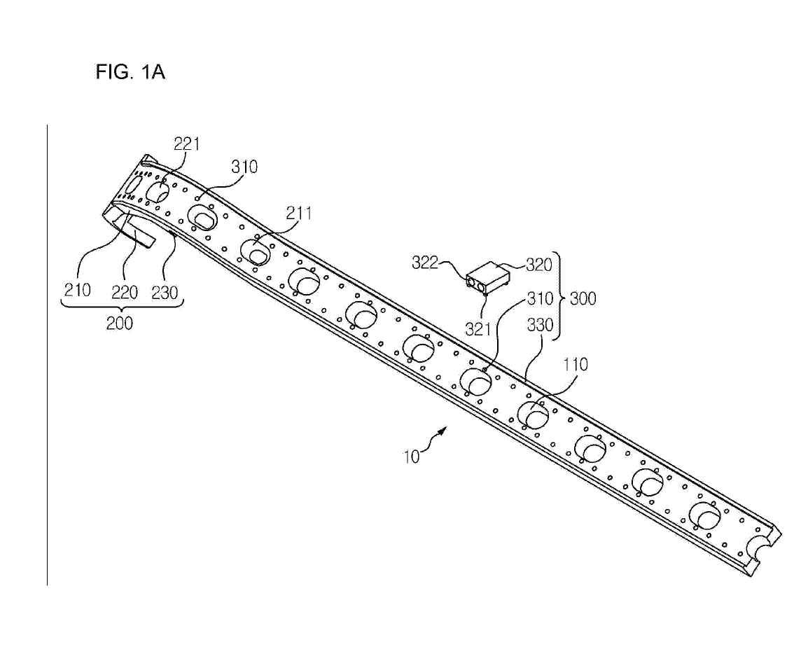

As shown in FIGS. 1 to 4, an adjustable implant 10 for fixing a femur according to a first embodiment of the present invention includes: a body 100 coming into contact with a side surface of a femur in a longitudinal direction of the femur; a head 200 extending from an upper end of the body 100, and being bent to be in a hook shape for being hooked over a greater trochanter of the femur; and a detachable wire locking part 300 detachably mounted the body 100 or to the head 200, and supporting a supporting the wire 400. The adjustable implant further includes a wire 400 wound around and fixing the femur by being coupled to the detachable wire locking part 300.

The body 100 has a predetermined thickness and is in a shape extending in a longitudinal direction, wherein the body 100 is provided with first locking holes 110 vertically penetrating therethrough at predetermined intervals. The body 100 may be made of titanium or titanium alloy to prevent deformation caused by shock during procedure, and preferably, the body 100 has a thickness of 1˜3 cm. Further, it is preferred that the body 100 is configured such that a lower surface thereof is in a curved shape so as to allow opposite edges of the lower surface come into close contact with the surface of the femur. The body 100 is longitudinally disposed to come into contact with a surface of a bone that extends from the greater trochanter of the femur, and then the wire is wound around the femur or locking pins are engaged with the first locking holes 110 to lock the body to the femur.

The head 200 includes: a bent part 210 extending from the upper end of the body 100 and being gently bent so as to come into contact with the greater trochanter of the femur; a hook 220 provided at an end of the bent part 210 to lock the implant by being hooked over the greater trochanter of the femur; and a fixing protrusion 230 provided on a rear surface of the bent part 210, and coming into close contact with a femur, thereby preventing the implant from moving on the femur. Further, the bent part 210 is provided with second locking holes 211 vertically penetrating therethrough at predetermined intervals to allow locking pins to be engaged therewith.

The head 200 is configured such that bent part 210 easily comes into contact with the greater trochanter of the femur, the hook 220 prevents the body 100 from moving down by being hooked over inside the greater trochanter of the femur, and the fixing protrusion 230 prevents the body 100 from moving from side to side by being hooked over the greater trochanter of the femur. Further, the locking pins are mounted through the second locking holes 211 to be directly locked to the greater trochanter of the femur or the wire is wound around the femur through the second locking holes 211.

Meanwhile, the detachable wire locking part 300 includes: base parts 310 provided on each of opposite sides of an upper surface of the body 100 or of the bent part 210 at predetermined intervals; and a wire locking member 320 being in a hexahedral shape, and having a coupling member 321 on a lower surface thereof so as to be detachably coupled to each of the base parts 310, with a pair of through-holes 322 penetrating through opposite sides of the wire locking member. Here, it is preferred that the base part 310 of the detachable wire locking part 300 includes a plurality of seat grooves being in a cylindrical shape and provided at predetermined intervals; and the coupling member 321 of the wire locking member 320 includes protrusions being in a cylindrical shape to be engaged with the seat grooves. It is further preferred that the protrusions are provided four in number.

Meanwhile, each of the body 100 and the bent part 210 is provided with a support part 330 on the upper surface thereof by protruding upward along a periphery of the base part 310 so as to support the opposite sides of the wire locking member 320. Here, it is preferred that the support part 330 is integrally formed to extend in a longitudinal direction.

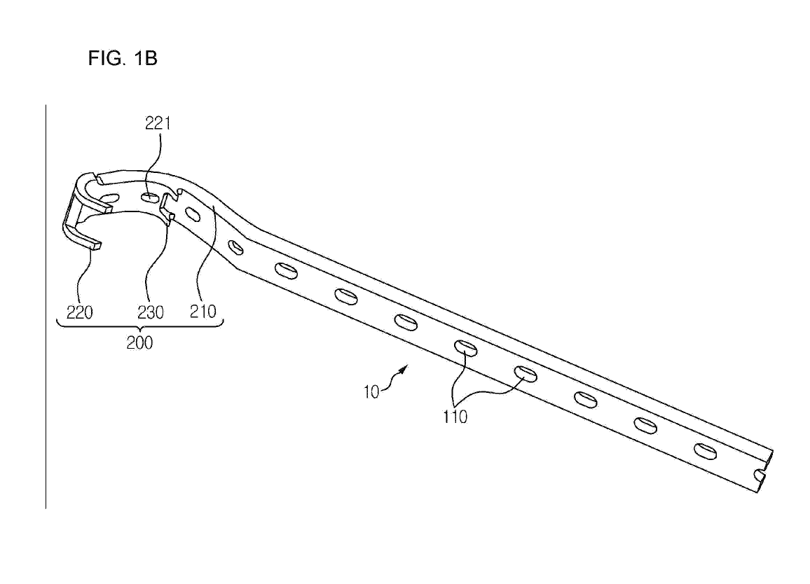

As shown in FIG. 2, thanks to the detachable wire locking part 300, when the wire is placed, the wire locking member 320 is coupled to the base part 310 that defines the location where the wire is to be placed, and thereby it is possible to accurately place the wire at a desired location.

Further, since the support part 330 comes into close contact with the opposite sides of the wire locking member 320, it is possible to prevent the wire locking member 320 from moving even when the wire that has been inserted into the through-holes 322 of the wire locking member is pulled.

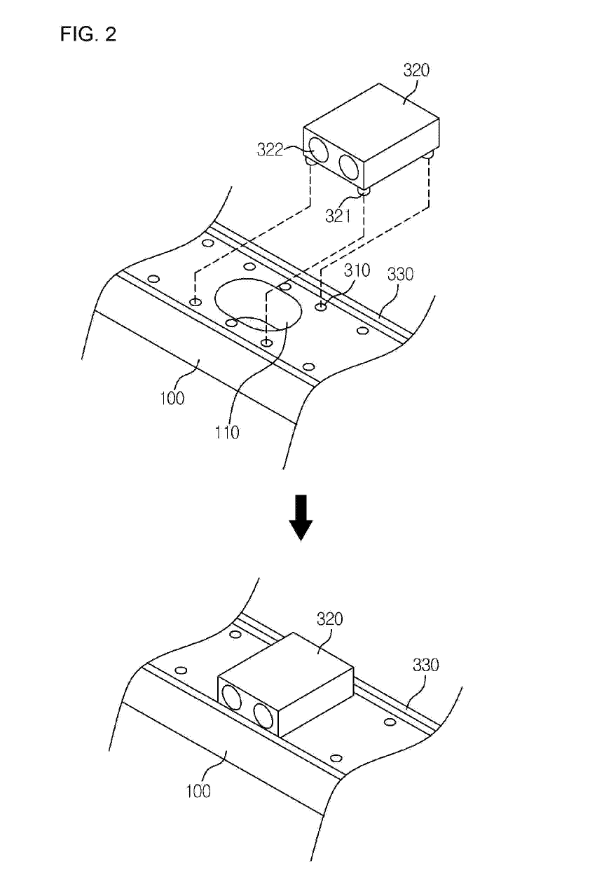

Meanwhile, as shown in FIG. 3, contact protrusions 500 protrude on each of inner surfaces of the through-holes 322 of the wire locking member 320. Here, it is preferred that the contact protrusions 500 are formed on the left and right inner surfaces of the through-holes, but the contact protrusions may be formed on the entire inner surfaces of the through-holes. Further, it is preferred that the contact protrusions are in a wedge shape inclined in one direction so as to prevent the wire 400 inserted thereinto from moving backwards. Thereby, when the wire locking member 320 is compressed by a tool, such as pliers after the wire 400 is inserted into the through-holes 322 of the wire locking member 320, the through-holes 322 are crushed such that the contact protrusions come into close contact with the wire 400 and are locked thereto.

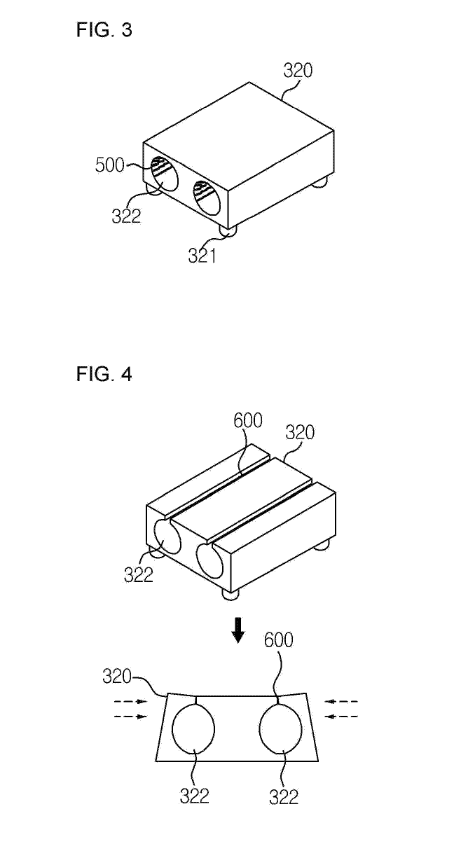

Further, as shown in FIG. 4, the wire locking member 320 is provided with slits 600 extending from an upper surface of the wire locking member to the through-holes 322. Accordingly, when the wire locking member 320 is compressed by a tool, such as pliers after the wire 400 is inserted into the through-holes 322 of the wire locking member 320, it is possible to compress the through-holes to pinch the wire with small effort.

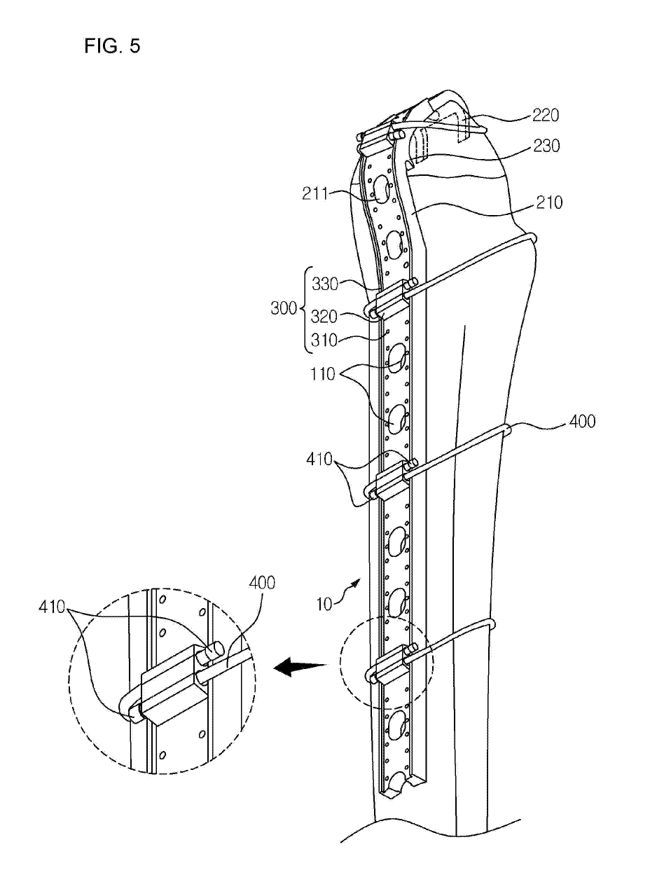

Referring to FIG. 5, the process of fixing the adjustable implant for fixing a femur of the present invention is as follows. Firstly, the bent part 210 of the head 200, which is bent at the upper end of the body 100, is placed over the greater trochanter of the femur, and the hook 220 provided at the end of the bent part 210 is hooked over the greater trochanter such that the implant is mounted to the femur. Here, to prevent the body 100 from moving, it is preferred that a locking means, such as a pin, is engaged with the second locking hole 211 formed in the bent part 210 or the first locking holes 110 formed in the body 100.

Further, in the state where the body 100 comes into close contact with the femur, the wire locking member 320 of the detachable wire locking part 300 is coupled to the base part 310 formed in the bent part 210 or the body 100, which is defined as a location where the wire 400 is required. After that, the wire 400 is inserted through a first end of one of the through-holes 322 formed through the wire locking member 320, then is wound around the femur, and then is inserted through a second end of a neighboring through-hole 322, and after that, the opposite ends of the wire 400 are pulled to securely come into close contact with the femur, and are cut thereby finishing the procedure. Here, it is preferred that the wire 400 is provided with a protection cover on a contact surface of the wire that comes into close contact with the femur so as to prevent the wire 400 from damaging the femur by coming into contact therewith.

Further, to prevent the wire 400 from slipping out of the through-holes 322, an upper portion of the wire locking member 320 is compressed by a tool, such as pliers, whereby the upper portion and the end portions of the wire locking member are crushed to pinch the wire 700 between inner surfaces of the through-holes 322. Here, it is possible to compress the through-holes 322 to pinch the wire 400 with small effort by providing the slits 600 extending from the upper surface of the wire locking member 320 to the through-holes 322. Further, in the case where the through-holes 322 are provided with the contact protrusions 500 on the inner surfaces thereof, the contact protrusions 500 are pressed into the surface of the wire 400 when the through-holes 322 are compressed, thereby preventing the wire 400 from moving backwards. Here, it is preferred that a protection cap 410 is put on each of opposite ends of the wire 400 to prevent sharp ends of the wire from being exposed outside by being unwound. The protection cap 410 serves to prevent the wire from being exposed, and also serves as a stopper by coming into close contact with the through-holes 322 when the wire is unwound.

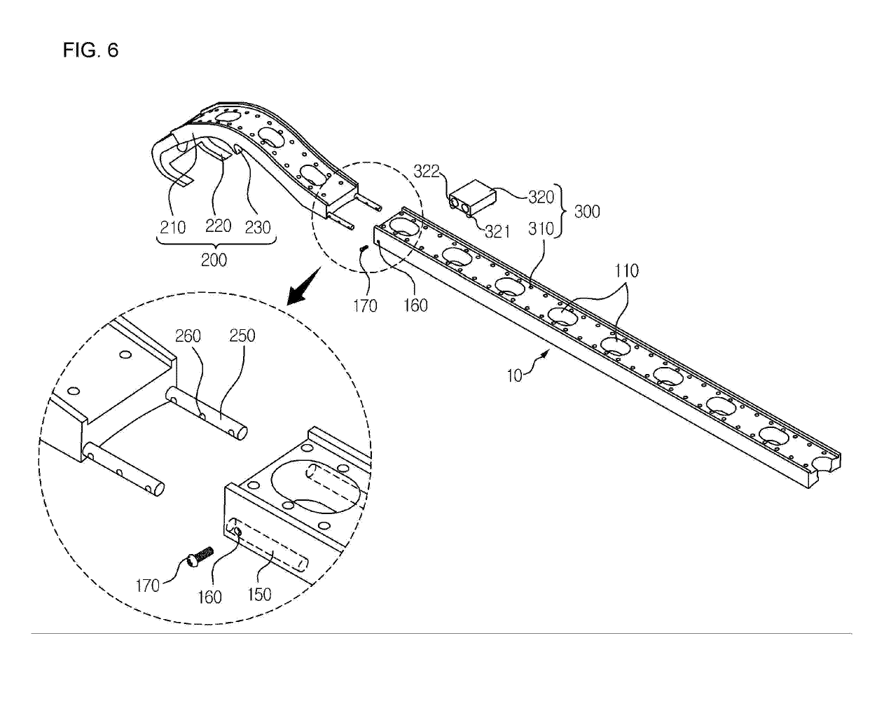

Meanwhile, as shown in FIG. 6, an adjustable implant for fixing a femur according to a second embodiment of the present invention includes: a body 100 coming into contact with a side surface of a femur in a longitudinal direction of the femur; a head 200 extending from an upper end of the body 100, and being bent to be in a hook shape for being hooked over a greater trochanter of the femur; and a detachable wire locking part 300 detachably mounted the body 100 or to the head 200, wherein the body 100 is detachably coupled to the head 200. Here, first locking holes 110 formed in the body 100, a bent part 210, a hook 220, and a fixing protrusion 230 formed in the head 200, and the detachable wire locking part 300 have the same features as the adjustable implant for fixing a femur according to the first embodiment, so the description thereof will be omitted.

Meanwhile, the body 100 includes: insertion grooves 150 provided at an upper end of the body; and locking grooves 160 provided on side surfaces of the body 100 and extending toward the insertion grooves 150. Here, it is preferred that the insertion grooves 150 and the locking grooves 160 are formed at lower opposite edges of the body so as not to overlap with the first locking holes 110 of the body.

Further, the head 200 includes connection protrusions 250 protrudingly provided at a lower end thereof so as to be engaged with the insertion grooves 150 of the body, with space adjusting grooves 260 being provided in the connection protrusions 250 at predetermined intervals. Here, the insertion grooves 150 of the body 100 and the connection protrusions 250 may be formed in various shapes, such as a cylindrical shape or a hexahedral shape, and may be two or four in number.

Accordingly, when the connection protrusions 250 of the head 200 are inserted into the insertion grooves 150 of the body 100, it is possible to adjust a gap between the head and the body. Then, after one of the space adjusting grooves 260 of the head are matched with the locking grooves 160 of the body, the adjusting screws 170 are inserted into the locking groove 160, and are engaged with the space adjusting grooves 260 of the head, thereby locking the head and the body.

The adjustable implant for fixing a femur according to the second embodiment allows the body and the head to be used separately or allows the length of the head coupled to the body to be adjusted.

Although a preferred embodiment of the present invention has been described for illustrative purposes, those skilled in the art will appreciate that various modifications, additions and substitutions are possible, without departing from the scope and spirit of the invention as disclosed in the accompanying claims.

Description of reference characters of important parts

| | |
|---|---|
| 100: body | 110: first locking holes |
| 200: head | 210: bent part |
| 211: second locking holes | 220: hook |
| 230: fixing protrusion | |
| 300: detachable wire locking part | |
| 310: base parts | 320: wire locking member |
| 321: coupling member | 322: through-holes |
| 330: support part | 400: wire |
| 500: contact protrusions | 600: slits |

What is claimed is:

1. An adjustable implant for fixing a femur, the adjustable implant comprising:
a body (100) provided with first locking holes (110) vertically penetrating therethrough at predetermined intervals;
a head (200) extending from an upper end of the body (100), and being bent to be in a hook shape for being hooked over a greater trochanter of a femur; and
a detachable wire locking part (300) detachably mounted to the body (100) or to the head (200), and supporting a wire (400), wherein
the detachable wire locking part (300) includes: base parts (310) provided on each of opposite sides of an upper surface of the body (100) or of a bent part (210) at predetermined intervals; and a wire locking member (320) being in a hexahedral shape, and having a coupling member (321) on a lower surface thereof so as to be detachably coupled to each of the base parts (310), with a pair of through-holes (322) penetrating through opposite sides of the wire locking member.

2. An adjustable implant for fixing a femur, the adjustable implant comprising:
a body (100) including: first locking holes (110) provided by vertically penetrating therethrough at predetermined intervals; insertion grooves (150) provided at an upper end of the body; and locking grooves (160) provided on side surfaces of the body (100) and extending toward the insertion grooves (150);
a head (200) being bent to be in a hook shape for being hooked over a greater trochanter of a femur, and including connection protrusions (250) protrudingly provided at a lower end thereof so as to be engaged with the insertion grooves (150) of the body, with space adjusting grooves (260) being provided in the connection protrusions (250) at predetermined intervals; and
a detachable wire locking part (300) detachably mounted to the body (100) or to the head (200), and supporting a wire (400), wherein
the detachable wire locking part (300) includes: base parts (310) provided on each of opposite sides of an upper surface of the body (100) or of a bent part (210) at predetermined intervals; and a wire locking member (320) being in a hexahedral shape, and having a coupling member (321) on a lower surface thereof so as to be detachably coupled to each of the base parts (310), with a pair of through-holes (322) penetrating through opposite sides of the wire locking member.

3. The adjustable implant for fixing a femur of claim 1, wherein
the body (100) is configured such that a lower surface thereof is in a curved shape so as to allow opposite edges of the lower surface come into close contact with a surface of a femur.

4. The adjustable implant for fixing a femur of claim 1, wherein the head (200) includes:
a bent part (210) extending from the upper end of the body (100) and being gently bent so as to come into contact with the greater trochanter of the femur;
a hook (220) provided at an end of the bent part (210) to lock the implant by being hooked over the greater trochanter of the femur; and
a fixing protrusion (230) provided on a rear surface of the bent part (210), and coming into close contact with a femur, thereby preventing the implant from moving on the femur.

5. The adjustable implant for fixing a femur of claim 4, wherein
the bent part (210) is provided with second locking holes (211) vertically penetrating therethrough at predetermined intervals.

6. The adjustable implant for fixing a femur of claim 1, wherein
the base part (310) of the detachable wire locking part (300) includes a plurality of seat grooves being in a cylindrical shape and provided at predetermined intervals; and the coupling member (321) of the wire locking member (320) includes protrusions being in a cylindrical shape to be engaged with the seat grooves.

7. The adjustable implant for fixing a femur of claim 1, wherein
each of the body (100) and the bent part (210) is provided with a support part (330) on the upper surface thereof by protruding upward along a periphery of the base part (310) so as to support the opposite sides of the wire locking member (320).

8. The adjustable implant for fixing a femur of claim 1, wherein
contact protrusions (500) protrude on each of inner surfaces of the through-holes (322) of the wire locking member (320).

9. The adjustable implant for fixing a femur of claim 1, wherein
the wire locking member (320) is provided with slits (600) extending from an upper surface of the wire locking member to the through-holes (322).

10. The adjustable implant for fixing a femur of claim 1, wherein a protection cap (410) is put on each of opposite ends of the wire (400) to prevent the wire from being unwound and being exposed outside.

11. The adjustable implant for fixing a femur of claim 2, wherein
the body (100) includes: the insertion grooves (150) provided at the upper end of the body; and the locking grooves (160) provided on the side surfaces of the body (100) and extending toward the insertion grooves (150), and
the head (200) includes the connection protrusions (250) provided protruding outwards from a location corresponding to the insertion grooves (150) at a lower end of the head, with the space adjusting grooves (260) being provided in the connection protrusions (250) at predetermined intervals.

12. The adjustable implant for fixing a femur of claim 2, wherein
the body (100) is configured such that a lower surface thereof is in a curved shape so as to allow opposite edges of the lower surface come into close contact with a surface of a femur.

13. The adjustable implant for fixing a femur of claim 2, wherein the head (200) includes:
a bent part (210) extending from the upper end of the body (100) and being gently bent so as to come into contact with the greater trochanter of the femur;
a hook (220) provided at an end of the bent part (210) to lock the implant by being hooked over the greater trochanter of the femur; and
a fixing protrusion (230) provided on a rear surface of the bent part (210), and coming into close contact with a femur, thereby preventing the implant from moving on the femur.

14. The adjustable implant for fixing a femur of claim 13, wherein
the bent part (210) is provided with second locking holes (211) vertically penetrating therethrough at predetermined intervals.

15. The adjustable implant for fixing a femur of claim 2, wherein
the base part (310) of the detachable wire locking part (300) includes a plurality of seat grooves being in a cylindrical shape and provided at predetermined intervals; and the coupling member (321) of the wire locking member (320) includes protrusions being in a cylindrical shape to be engaged with the seat grooves.

16. The adjustable implant for fixing a femur of claim 2, wherein
each of the body (100) and the bent part (210) is provided with a support part (330) on the upper surface thereof by protruding upward along a periphery of the base part (310) so as to support the opposite sides of the wire locking member (320).

17. The adjustable implant for fixing a femur of claim 2, wherein
contact protrusions (500) protrude on each of inner surfaces of the through-holes (322) of the wire locking member (320).

18. The adjustable implant for fixing a femur of claim 2, wherein
the wire locking member (320) is provided with slits (600) extending from an upper surface of the wire locking member to the through-holes (322).

19. The adjustable implant for fixing a femur of claim 2, wherein a protection cap (410) is put on each of opposite ends of the wire (400) to prevent the wire from being unwound and being exposed outside.
